(19) United States
(12) Patent Application Publication
(10) Pub. No.: US 2024/0271106 A1
(43) Pub. Date: Aug. 15, 2024

(54) RECOMBINANT NEWCASTLE DISEASE VIRUS AND PREPARATION METHOD THEREFOR, RECOMBINANT PLASMID, AND USE THEREOF

(71) Applicant: JIANGSU KANIONREAL BIOMEDICAL TECHNOLOGY CO., LTD, Lianyungang, Jiangsu (CN)

(72) Inventors: Wei XIAO, Lianyungang (CN); Deshan LI, Lianyungang (CN); Tianyan LIU, Lianyungang (CN); Zhihang LIU, Lianyungang (CN); Zhenzhong WANG, Lianyungang (CN); Shan JIANG, Lianyungang (CN); Xiaolian HE, Lianyungang (CN); Xuehong DONG, Lianyungang (CN); Yanqiu WANG, Lianyungang (CN)

(73) Assignee: JIANGSU KANIONREAL BIOMEDICAL TECHNOLOGY CO., LTD, Lianyungang, Jiangsu (CN)

(21) Appl. No.: 17/914,882

(22) PCT Filed: May 21, 2021
(86) PCT No.: PCT/CN2021/095200

§ 371 Date: Sep. 27, 2022

(30) Foreign Application Priority Data
Mar. 30, 2020 (CN) 202010238162.4

(51) Int. Cl. C12N 7/00; A61K 35/768; A61P 35/00; C12N 15/85

(52) U.S. Cl. CPC C12N 7/00; A61K 35/768; A61P 35/00; C12N 15/85; C12N 2760/18121; C12N 2760/18132; C12N 2760/18152; C12N 2800/107

(57) ABSTRACT

A recombinant Newcastle disease virus and a preparation method therefor, a recombinant plasmid, and use of the recombinant Newcastle disease virus or the recombinant plasmid. The recombinant Newcastle disease virus is obtained by replacing an F protein of a Newcastle disease virus lasota with an F protein encoded by a DNA as set forth in SEQ ID NO: 1 at positions 7274-8935 from a 5′-terminus. The recombinant Newcastle disease virus is safe and capable of effectively inhibiting tumour cells and promoting tumour cell apoptosis.

### TECHNICAL FIELD

The present application in general relates to the field of biotechnologies, in particular to a recombinant Newcastle disease virus and a preparation method therefor, a recombinant plasmid, and use thereof.

### BACKGROUND

Major means for treating cancers include surgeries, radiotherapies, chemotherapies, immunotherapies, monoclonal antibody therapies, and viral vaccines.

Malignant tumours are one of the major diseases endangering human's health. According to the data in 2015 Global Cancer Statistics, there were about 14.1 million new cancer cases and up to 8.2 million cancer deaths across the world in 2015. It is predicted that by 2025, the number of new cancer cases across the world every year will reach up to 24.49 million. For a long time, the methods adopted by people to treat cancers mainly include traditional surgical resections, radiotherapies, and chemotherapies. These methods can control the development of tumours to some extent, but showing limited efficacy for patients with advanced tumour spread, and at the same time, causing severe trauma to the normal cells of a human body. Therefore, there is an urgent need of a new method for treating a malignant tumour. An oncolytic virus therapy is a novel tumour treatment method by which a tumour is killed by selectively infecting tumour cells with viruses. As an important part of tumour immunity, it has a great advantage as compared with other tumour immunotherapies. The oncolytic virus therapy has undergone three stages of development, and has achieved great results. At present, the anti-tumour effect can be enhanced by carrying exogenous genes, among which T-vec carrying GM-CSF exhibits a good efficacy and was approved by the US FDA in 2015 for the local treatment of an unresectable melanoma that recurred for the first time. In December of the same year, T-vec was further approved by the European Union, indicating a great development potential of oncolytic viruses.

In 1950s, it was found that Newcastle disease viruses (NDV) were capable of inhibiting the metastasis of gastric cancer. Therefore, NDV can be used as an emerging tumour bio-therapeutic factor, and its application to tumour treatment has been studied deeper and deeper due to its high targeting property and the maturity of molecular biotechnologies. As a single negative-stranded RNA virus, NDV encodes six types of structural proteins (L, NP, P, HN, F, and M).

Compared with other oncolytic viruses, the Newcastle disease virus has a natural targeting property against tumour cells, and broad-spectrum anti-tumour properties, showing greater advantages in the anti-tumour aspect. However, how to improve its oncolytic effect and to ensure its safety is still a huge challenge for us to be confronted with.

### SUMMARY

An object of the present application is to provide a recombinant Newcastle disease virus and a preparation method therefor, a recombinant plasmid, and use thereof.

The present application provides a recombinant Newcastle disease virus obtained by replacing an F protein of a Newcastle disease virus lasota with an F protein of a Newcastle disease virus mesogenic strain.

Optionally, according to the recombinant Newcastle disease virus mentioned above, a coding DNA of an F protein of the recombinant Newcastle disease virus is as set forth in SEQ ID NO: 1 at positions 7274-8935 from a 5′-terminus.

Optionally, according to the recombinant Newcastle disease virus mentioned above, the recombinant Newcastle disease virus is obtained by replacing an F gene in a genome of the Newcastle disease virus lasota with an F gene of the Newcastle disease virus mesogenic strain. Preferably, a DNA corresponding to the F gene of the recombinant Newcastle disease virus is as set forth in SEQ ID NO: 1 at the positions 7274-8935 from the 5′-terminus. More preferably, a DNA corresponding to a genome of the recombinant Newcastle disease virus is as set forth in SEQ ID NO: 1.

Optionally, according to the recombinant Newcastle disease virus mentioned above, a DNA corresponding to a genome of the recombinant Newcastle disease virus further comprises an exogenous gene, which is selected from one or more of a group consisting of DR5, TRAIL, hIL2, P53, PD1, CD, and mIL12.

Preferably, the exogenous gene is DR5, TRAIL, hIL2, P53, PD1, CD, or mIL12.

Preferably, the exogenous gene is mIL12-hIL2, P53-hIL2, PD1-hIL2, or DR5-TRAIL.

Preferably, the DR5 is as set forth in SEQ ID NO: 2, the TRAIL is as set forth in SEQ ID NO: 3, the hIL2 is as set forth in SEQ ID NO: 4, the P53 is as set forth in SEQ ID NO: 5, the PD1 is as set forth in SEQ ID NO: 6, the CD is as set forth in SEQ ID NO: 7, and the mIL 12 is as set forth in SEQ ID NO: 8.

Preferably, the exogenous gene is located between a P gene and an M gene of the recombinant Newcastle disease virus.

The present application further provides a recombinant plasmid obtained by replacing an F gene in a pBrlasota plasmid, wherein the F gene of the recombinant plasmid is as set forth in SEQ ID NO: 1 at positions 7274-8935 from a 5′-terminus.

Optionally, according to the recombinant plasmid mentioned above, the recombinant plasmid is a DNA molecule plasmid as set forth in SEQ ID NO: 1.

Optionally, according to the recombinant plasmid mentioned above, the recombinant plasmid further comprises an exogenous gene, which is selected from one or more of a group consisting of DR5, TRAIL, hIL2, P53, PD1, CD, and mIL12.

Preferably, the exogenous gene is DR5, TRAIL, hIL2, P53, PD1, CD, or mIL12.

Preferably, the exogenous gene is mIL12-hIL2, P53-hIL2, PD1-hIL2, or DR5-TRAIL.

Preferably, the DR5 is as set forth in SEQ ID NO: 2, the TRAIL is as set forth in SEQ ID NO: 3, the hIL2 is as set forth in SEQ ID NO: 4, the P53 is as set forth in SEQ ID NO: 5, the PD1 is as set forth in SEQ ID NO: 6, the CD is as set forth in SEQ ID NO: 7, and the mIL 12 is as set forth in SEQ ID NO: 8.

Preferably, the exogenous gene is located between a P gene and an M gene of a genome of the recombinant Newcastle disease virus.

The present application further provides a preparation method for a recombinant Newcastle disease virus, comprising transfecting the recombinant plasmid mentioned above to cells or cell line for culturing to obtain the recombinant Newcastle disease virus.

Optionally, according to the preparation method mentioned above, the recombinant plasmid and a helper plasmid are co-transfected to cells or cell line for culturing. The cell is a mammalian cell. The helper plasmid is, for example, a pBL-N plasmid, a pBL-P plasmid, or a pBL-L plasmid.

The present application further provides use of the recombinant Newcastle disease virus mentioned above or the recombinant plasmid mentioned above in preparation of a drug. The drug has the following function(s) of (a) and/or (b) and/or (c): (a) treating a tumour; (b) inhibiting tumour cell proliferation; and (c) killing tumour cells.

The present application further provides a drug, comprising the recombinant Newcastle disease virus mentioned above, and/or the recombinant plasmid mentioned above. The drug has the following function(s) of (a) and/or (b) and/or (c): (a) treating a tumour; (b) inhibiting tumour cell proliferation; and (c) killing tumour cells.

Optionally, the tumour is selected from one or more of a group consisting of a liver cancer, a breast cancer, a non-small cell lung cancer, a melanoma, a neuroblastoma, a lung cancer, a pancreatic cancer, a thyroid cancer, a kidney cancer, a glioma, a myosarcoma, an esophageal cancer, a uterine cancer, and a colorectal cancer.

Optionally, the tumour cell is selected from one or more of a group consisting of a liver cancer cell, a breast cancer cell, a non-small cell lung cancer cell, a melanoma cell, a neuroblastoma cell, a lung cancer cell, a pancreatic cancer cell, a thyroid cancer cell, a kidney cancer cell, a glioma cell, a myosarcoma cell, an esophageal cancer cell, an uterine cancer cell, and a colorectal cancer cell.

In the present application, virus strains having a stronger effect of promoting cell fusion are screened out; the structural protein genes of the strains are acquired; structural protein genes of lasota are replaced by means of a reverse genetic manipulation technique and a molecular cloning technique; and exogenous genes are further inserted to successfully construct and rescue recombinant chimeric viruses. The recombinant chimeric viruses are continuously passaged in chicken embryos for 10 generations. The results of a proliferation stability experiment have demonstrated that the recombinant Newcastle disease virus could proliferate continuously and stably in the chicken embryos, and the effects of the recombinant chimeric virus on killing tumour cells are systematically compared.

The recombinant chimeric virus according to the present application has good safety and is capable of effectively inhibiting tumour cells, promoting tumour cell apoptosis, and effectively treating a tumour.

### BRIEF DESCRIPTION OF THE DRAWINGS

FIG. 1 shows the results of a virus strain screening test according to Example 1;

FIG. 2 shows the results of an exogenous gene expression position determination test according to Example 2;

FIG. 3A to FIG. 3E show the killing effects of a recombinant chimeric virus against tumour cells as detected in a cell fusion experiment according to Example 4;

FIG. 4 shows the killing effect of a recombinant chimeric virus against HepG-2 cells as detected by an MTT method according to Example 5;

FIG. 5 shows the killing effect of the recombinant chimeric virus against Caki-1 cells as detected by the MTT method according to Example 5;

FIG. 6 shows the killing effect of the recombinant chimeric virus against A549 cells as detected by the MTT method according to Example 5;

FIG. 7 shows the killing effect of the recombinant chimeric virus against SK-MEL-5 cells as detected by the MTT method according to Example 5;

FIG. 8 shows the killing effect of the recombinant chimeric virus against LN229 cells as detected by an MTT method according to Example 5;

FIG. 9 shows the killing effect of the recombinant chimeric virus against liver cancer cells as detected by an Annexin V/PI method according to Example 5;

FIG. 10 shows the statistics of tumour volume after H22 tumour-bearing mice are treated with a recombinant chimeric virus according to Example 6;

FIG. 11 shows the statistics of tumour weight after the H22 tumour-bearing mice are treated with the recombinant chimeric virus according to Example 6;

FIG. 12 shows the statistics of survival rate of the H22 tumour-bearing mice treated with the recombinant chimeric virus according to Example 6; and

FIG. 13 shows the pathological observations of tumour tissues after the H22 tumour-bearing mice are treated with the recombinant chimeric virus according to Example 6.

### DETAILED DESCRIPTION OF THE EMBODIMENTS

Specific embodiments of the present application will be illustrated below in further detail in combination with accompanying drawings and examples for a better understanding of the solutions of the present application as well as the advantages thereof in various aspects. Nevertheless, the specific embodiments and examples described below are merely for an illustrative purpose, and are not intended to limit the present application.

The experimental methods in the examples below are conventional methods, unless otherwise stated. Test materials used in the examples below are purchased from common biochemical reagent stores, unless otherwise stated. For quantitative tests in the examples below, three repeated experiments are set up, and the results are averaged. Unless otherwise specified in the following examples, the technical means used in these examples are conventional means well-known to a person skilled in the art or common commercially-available instruments and reagents. References can be made to Molecular Cloning: A Laboratory Manual (Third Edition) (Science Press), Microbiological Experiments (Fourth Edition) (Higher Education Press), and the manufacturer's instructions for use for the corresponding instruments and reagents, etc.

A reference mentioning “pBrlasota plasmid” is “Identification of Optimal Insertion Site in Recombinant Newcastle Disease Virus (rNDV) Vector Expressing Foreign Gene to Enhance Its Anti-Tumour Effect” (Ziye Pan, PLOS ONE.; Harbin Boxiang Medical Technology Development Co., Ltd.).

A reference mentioning “pBL-N plasmid”, “pBL-P plasmid”, and “pBL-L plasmid” is “Genetically engineered Newcastle disease virus expressing interleukin 2 is a potential drug candidate for cancer immunotherapy” (Fuliang Bai, Immunology letters; Harbin Boxiang Medical Technology Development Co., Ltd.).

The pBrlasota plasmid contains the NP, P, M, F, HN, and L genes of the Newcastle disease virus, with PmeI and SacII digestion recognition sites between the P gene and the M gene. The pBrlasota, pBL-N, pBL-P, and pBL-L plasmids are co-transfected to mammalian cells and cultured (with the pBL-N, pBL-P, and pBL-L plasmids as helpers, and the pBrlasota plasmid providing a virus whole-genome) to obtain a virus strain lasota.

The exogenous genes involved herein include a death receptor gene DR5, a tumour necrosis factor-related apoptosis-inducing ligand gene TRAIL, a human interleukin gene 2hIL2, a cancer suppressor gene P53, a programmed death receptor 1 gene PD1, a suicide gene CD, and a murine interleukin gene 12mIL12.

pMD19-T: purchased from TaKaRa Engineering (Dalian) Co., Ltd. (Dalian TaKaRa Bio Inc.).

BHK-21 cells (baby hamster kidney cells): purchased from ATCC.

Liver cancer cells HepG2, kidney cancer cells Caki-1, non-small cells lung cancer cells A549, neuroblastoma cells LN229, melanoma cells SK-MEL5, liver cancer cells HCCLM3, melanoma cells SK-MEL2, pancreatic cancer cells ASPC-1, thyroid cancer cells FTC133, kidney cancer cells 786-O, neuroblastoma cells U87-MG, myosarcoma cells HT1080, esophageal cancer cells KYSE-150, uterine cancer cells Hela, breast cancer cells MCF7, and colorectal cancer cells HCT116 were purchased from the Cell Resource Center of Shanghai Institutes for Biological Sciences, Chinese Academy of Sciences.

High-glucose DMEM medium, trypsin, new born calf serum (NBCS), and fetal bovine serum (FBS): purchased from GIBCO.

SPF chicken embryo: purchased from Harbin Veterinary Research Institute, Chinese Academy of Agricultural Sciences.

Balb/c mice (Kunming mice): purchased from Vital River Laboratories, Beijing.

### Example 1: Virus Strain Screening

Chicken embryo fibroblasts in a logarithmic growth phase were digested with trypsin and collected, and then seeded into a 6-well plate. Three virulent Newcastle disease virus strains F48E9, Turkey/US(ND)/43084/92 (TkND) and Game fowl/US(CA)/211472/02 (CA02) and one mesogenic strain Anhinga (the strains were donated by Southeast Poultry Research Laboratory and stored in our laboratory, and a reference mentioning the virus strains included “Evaluation of Newcastle disease virus chimeras expressing the Hemagglutinin-Neuraminidase protein of velogenic strains in the context of a mesogenic recombinant virus backbone”. Virus Research, Volume 129, Issue 2, November 2007, Pages 182-190) infected cells at 0.1 MOI respectively, and a control group was a virus-free SPF chicken embryo allantoic fluid. After 24 h, the 6-well plate were taken out and observed under an inverted fluorescence microscope for a cell fusion effect. By analysing a syncytial formation capability, virus strains having strong fusion capability and weak virulence were screened out for subsequent experiments. The results were shown in FIG. 1. Among the four virus strains, F48E9 showed the strongest fusion-promoting capability, and the strain Anhinga shows a stronger fusion-promoting capability and the weakest virulence.

### Example 2: Preparation of Recombinant Chimeric Virus

### I. Construction of Recombinant Plasmid (Taking F Gene Replacement as an Example)

1. The virus strain Anhinga with a stronger fusion-promoting effect and weaker virulence was screened out, and the sequence of its F gene was acquired from NCBI (GenBank: AAS67135.1), and overlap PCR primers were designed.
2. Taking a pAnh plasmid (the involved reference was “Evaluation of Newcastle disease virus chimeras expressing the Hemagglutinin-Neuraminidase protein of velogenic strains in the context of a mesogenic recombinant virus backbone”) as a template, an F gene fragment was amplified by PCR by using F gene specific primers, and linked to pMD19-T; the linking product was transformed into Escherichia coli DH5α; the plasmid was extracted and identified; and the correctly identified pMD19-T-F plasmid was used for the next experiment.
3. With the PCR technique, the target fragments A and B were amplified by using the pBrlasota plasmid as a template (primers for the fragment A: 5′ CACGTG AAAGCGCCAGAGAAGATTCCCGGGA 3′; P2: 5′ TTTGGGGCCCATCTTGCACCTGGAGGGCGCCAACCGG 3′; P3: in TCCAGGTGCAAGATGGGCCCCAAACCCCCCACCGGAA 3′. B fragment primers: P6: 5′ CCTCATCTGTGT TCATGTTTTTGTGGTGGCTCTCATC 3′; P7: 5′ ACAAAAACATGAACACAGATGAGGAACGAAGGTTTCC 3′; and P8: 5′ GGCCTGAGAGGCCACGCGTCGCCGCGGGCCGGTT 3′). The target fragment F was amplified by using the pMD19-T-F plasmid as a template, and the fragments A, F and B were extracted by gel. Then, the fragment A-F-B of interest was amplified by the overlap PCR method using the purified fragments A, F and B as templates, and linked to a pMD19-T vector; the linking product was transformed into Escherichia coli DH5α; plasmids were extracted and identified; and the correctly identified plasmids were named as pMD19-T-A-F-B, which carry two digestion sites PmlI and SfiI.
4. The plasmids pMD19-T-A-F-B from step 3 were digested using restriction enzymes PmlI and SfiI, and the cleaved product was extracted by gel.
5. The pBrlasota plasmid was digested using the restriction enzymes PmlI and SfiI, and the vector was extracted by gel.
6. The digested product from step 4 and the vector from step 5 were linked to obtain a recombinant chimeric plasmid pBrlasota-F. PCR and double digestion identification were performed on the recombinant chimeric plasmid; the correctly identified plasmid samples were sub-packaged and sent to Shanghai Sangon for sequencing. The sequencing results were aligned using sequence analysis software DNAMAN; and the plasmid with the sequencing result consistent with a sequence of interest was named as pBrlasota-F. The nucleotide sequence of the recombinant chimeric plasmid pBrlasota-F was as set forth in SEQ ID NO:1 of the sequence listing (the nucleotide sequence at positions 7274-8935 from the 5′-terminus in SEQ ID NO: 1 was the nucleotide sequence of the F gene).

Other recombinant chimeric plasmids were constructed in the same way: pBrlasota-HN (HN gene sequence, GenBank: AAS67136.1) pBrlasota-P (P gene sequence in GenBank: AAS67133.1), pBrlasota-M (M gene sequence in GenBank: AAS67134.1).

### II. Determination of Optimal Insertion Position for Exogenous Gene

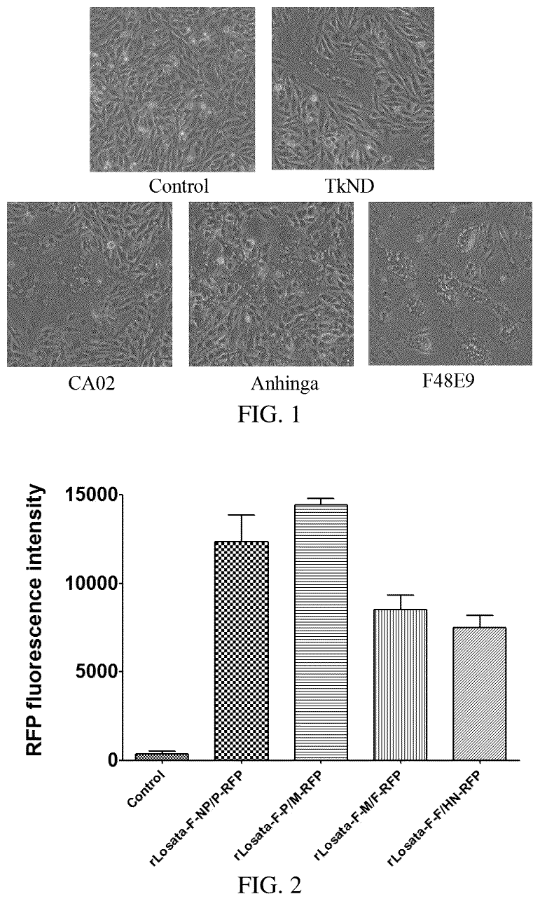

1. Restriction Enzyme cutting sites were respectively introduced to the NP/P, P/M, M/F, and F/HN sites of the pBrlasota-F plasmid using a molecular biology method. Recombinant plasmids pBrlasota-F-NP/P (AscI, SfiI), pBrlasota-F-P/M (SacII, PmeI), pBrlasota-F-M/F (HpaI, MluI), and pBrlasota-F-F/HN (HpaI, MluI) were obtained.
2. RFP gene fragments carrying different restriction enzyme cutting sites were amplified by PCR using specific primers for a red fluorescent protein RFP gene (red fluorescent protein RFP gene sequence in GenBank: MG719763.1), and linked to a pMD19-T vector; the linking product was transformed into Escherichia coli DH5α; and the plasmid was extracted and identified as carrying different restriction enzyme cutting sites.
3. The plasmid from step 2 was digested using a restriction enzyme, and the cleaved product was extracted by gel.
4. The recombinant plasmid from step 1 was digested using a restriction enzyme, and the vector was extracted by gel.
5. The digested product from step 3 and the vector from step 4 were linked to obtain recombinant plasmids pBrlasota-F-NP/P-RFP, pBrlasota-F-P/M-RFP, pBrlasota-F-M/F-RFP, and pBrlasota-F-F/HN-RFP.
6. BHK-21 cells were co-transfected with the recombinant plasmid from step 5 and the helper plasmid to obtain recombinant virus rlasota-F-NP/P-RFP, rlasota-F-P/M-RFP, rlasota-F-M/F-RFP, and rlasota-F-F/HN-RFP.
7. MCF7 cells in the logarithmic growth phase were digested with trypsin and then collected, and seeded into a 6-well plate. The cells were respectively infected with the recombinant chimeric viruses rlasota-F-NP/P-RFP, rlasota-F-P/M-RFP, rlasota-F-M/F-RFP, and rlasota-F-F/HN-RFP at 1 MOI, the parental virus rlasota was used as a control, and the control group was virus-free SPF chicken embryo allantoic fluid. After 24 h, the 6-well plate was taken out, and the average fluorescence intensity of the cells infected with respective virus was detected by the flow cytometry. The results were shown in FIG. 2, showing the strongest red fluorescence intensity between P and M sites, which indicates the highest RFP expression level between the P and M sites.

### III. Construction of Recombinant Plasmid Inserted with Exogenous Gene (Taking PD1 Gene Insertion as an Example)

1. Monocytes were isolated from human peripheral blood cells, and RNA was extracted and reverse-transcribed into cDNA. According to the human PD1 sequence (AY238517.1) that has been published in GenBank, PCR primers P1 and P2 were designed using Primer Premier 5.0 software to clone the complete PD1 gene. Pme I and Sac II digestion sites were designed at both ends of the primers to match the pBrlasota-F vector plasmid.
2. The PCR product from step 1 was linked to pMD19-T and transformed into Escherichia coli; plasmids were extracted and identified; and the correctly identified pMD19-T-PD1 plasmids were used for the next experiment.
3. The pMD19-PD1 plasmids from step 2 were digested using restriction enzymes Pme I and Sac II, and the cleaved product was extracted by gel.
4. The pBrlasota-F plasmids were digested using restriction enzymes Pme I and Sac II, and the vector was extracted by gel.
5. The cleaved product from step 3 was linked to the vector from step 4 to obtain a recombinant chimeric plasmid pBrlasota(F)-PD1, with the PD1 gene inserted between the P and M genes of the plasmid. PCR as well as Pme I and Sac II double digestion identification were performed on the recombinant chimeric plasmid; the correctly identified plasmid samples were sub-packaged and sent to Shanghai Sangon for sequencing. The sequencing results were aligned using sequence analysis software DNAMAN; and the sequenced result was consistent with a sequence of interest. The PD1 nucleotide sequence of the recombinant chimeric plasmid pBrlasota(F)-PD1 was as set forth in SEQ ID NO. 6 in the sequence listing.

Other recombinant chimeric plasmids inserted with exogenous genes were constructed using the same method. The constructed recombinant chimeric plasmids were specifically as follows: pBrlasota(F)-DR5 (a DR5 sequence as set forth in SEQ ID NO: 2), pBrlasota(F)-TRAIL (a TRAIL sequence as set forth in SEQ ID NO: 3), pBrlasota(F)-hIL2 (a hIL2 sequence as set forth in SEQ ID NO: 4), pBrlasota(F)-P53 (a P53 sequence as set forth in SEQ ID NO:5), pBrlasota(F)-CD (a CD sequence as set forth in SEQ ID NO: 7), pBrlasota(F)-mIL12 (a mIL12 sequence as set forth in SEQ ID NO: 8), pBrlasota(F)-DR5-TRAIL (i.e., a DR5-TRAIL combination as the exogenous gene), pBrlasota(F)-mIL12-hIL2 (i.e., a mIL12-hIL2 combination as the exogenous gene), pBrlasota(F)-P53-hIL2 (i.e., a P53-hIL2 combination as the exogenous gene), and pBrlasota(F)-PD1-hIL2 (i.e., a PD1-hIL2 combination as the exogenous gene).

### IV. Preparation of Recombinant Chimeric Virus

1. BHK-21 cells were co-transfected respectively with the plasmid pBrlasota, the recombinant chimeric plasmids pBrlasota-HN, pBrlasota-F, pBrlasota-P, pBrlasota-M, pBrlasota(F)-DR5, pBrlasota(F)-TRAIL, pBrlasota(F)-hIL2, pBrlasota(F)-P53, pBrlasota(F)-PD1, pBrlasota(F)-CD, pBrlasota(F)-mIL12, pBrlasota(F)-DR5-TRAIL, pBrlasota(F)-mIL12-hIL2, pBrlasota(F)-P53-hIL2 and pBrlasota(F)-PD1-hIL2, together with the helper plasmids pBL-NP, pBL-P and pBL-L (about 1×106 cells transfected with 2 μg of recombinant chimeric plasmids, 1 μg of pBL-N plasmids, 0.5 μg of pBL-P plasmids and 0.25 μg of pBL-L plasmids). The transfected cells were placed in a 5% CO2 environment at 37° C. for static culture for 72 h.
2. The transfected cells obtained in step 1 were repeatedly frozen and thawed three times, centrifuged to collect the cell supernatant, then seeded into the allantoic cavities of 9-11-day-old SPF chicken embryos. The chicken embryo allantoic cavities were cultured for 72 h in an environment at 37° ° C., and the chicken embryo allantoic fluid was collected for HA detection.
3. The HA-positive chicken embryo allantoic liquid obtained in step 2 was passaged, seeded into the allantoic cavities of fresh 9-11-day-old SPF chicken embryos, and cultured for 72 h in an environment at 37° ° C. The chicken embryo allantoic liquid was collected for HA detection. The HA-positive chicken embryo allantoic liquid was passaged for 10 generations, and the proliferation stability of the recombinant viruses was detected.
4. The chicken embryo allantoic liquid obtained from step 3 was taken for mass reproduction of the viruses, then seeded into the allantoic cavities of new 9-11-day-old SPF chicken embryos, and cultured for 72 h in an environment at 37° C. The chicken embryo allantoic liquid was collected for HA detection. The allantoic liquid with the HA titer greater than 29 was selected, mixed and sub-packaged for later use.
5. The allantoic liquid was collected from each group for Western Blot to detect the expression of each exogenous gene. The results showed that, compared with the rlasota group, the recombinant chimeric viruses expressing the exogenous gene was capable of stably expressing the exogenous gene.

In the following description, unless otherwise specified, the recombinant chimeric viruses, the chimeric viruses and the recombinant viruses refer to the recombinant chimeric viruses prepared by the above methods. rlasota-HN, rlasota-F, rlasota-P, rlasota-M, rlasota(F)-DR5, rlasota(F)-TRAIL, rlasota(F)-hIL2, rlasota(F)-P53, rlasota(F)-PD1, rlasota(F)-CD, rlasota(F)-mIL12, rlasota(F)-DR5-TRAIL, rlasota(F)-mIL12-hIL2, rlasota(F)-P53-hIL2 and rlasota(F)-PD1-hIL2 respectively refer to the recombinant chimeric viruses prepared with the above methods by using the recombinant chimeric plasmids pBrlasota-HN, pBrlasota-F, pBrlasota-NP, pBrlasota-M, pBrlasota(F)-DR5, pBrlasota(F)-TRAIL, pBrlasota(F)-hIL2, pBrlasota(F)-P53, pBrlasota(F)-PD1, pBrlasota(F)-CD, pBrlasota(F)-mIL12, pBrlasota(F)-DR5-TRAIL, pBrlasota(F)-mIL12-hIL2, pBrlasota(F)-P53-hIL2 and pBrlasota(F)-PD1-hIL2.

In the following description, unless otherwise specified, the parental viruses refer to the Newcastle disease viruses rlasota and Anhinga.

### Example 3: Determination of Virulence and Pathogenicity of Recombinant Chimeric Viruses

### I. Determination of EID50

The rescued recombinant chimeric viruses or parental viruses were serially diluted 10 times with sterilized PBS respectively. Chicken embryos were taken out from an incubator at 37° ° C. Six 9-11-day-old SPF chicken embryos were seeded with the viruses at each dilution (100 μL for each), and incubated in the incubator at 37ºC for 4-6 days. Each chicken embryo needed to be detected by HA assay. The number of embryos infected at each dilution was recorded. The EID50 of each of the recombinant chimeric viruses or parental viruses was calculated according to a Reed and Muench method.

### II. Determination of TCID50

1. DF-1 cells were seeded into a 96-well microplate by using a DMEM medium containing 10% FBS and 1% antibiotics, and held in a 5% CO2 incubator at 37° C. overnight.
2. Before the recombinant chimeric viruses and the parental viruses were seeded, the original cell culture medium added in step 1 was discarded, and 180 μL of fresh DMEM culture medium containing 10% allantoic fluid and 1% antibiotics.
3. 20 μL of the recombinant chimeric viruses or parental viruses was seeded into the top row of cell wells; the mixture was gently pipetted up and mixed; and 20 μL of the mixture was pipetted to the lower cell wells for 10-fold serial dilution. Three replicates were set up for each virus.
4. In the 5% CO2 cell incubator at 37° C., the recombinant chimeric viruses or parental viruses were seeded and incubated for 1 h; then, the culture medium added in step 3 was discarded, and the residue was washed with PBS; and then, 200 μL of fresh DMEM culture medium containing 10% allantoic fluid and 1% antibiotics was added.
5. The culture continued in a 5% CO2 cell incubator at 37° C. After 72 h, the wells with cytopathy were observed under an optical inverted microscope, the number of wells with cytopathy was recorded, and the titer of the respective virus was calculated according to the Reed and Muench method.

### III. Determination of MDT

The recombinant chimeric viruses or parental viruses were serially diluted 10 times with sterilized PBS respectively. The viruses subjected to doubling dilution were seeded to six 10-day-old SPF chicken embryos (100 μL for each) via allantoic cavities, and were continuously cultured for 7 days in the incubator at 37° C. The death time and death number of the chicken embryos seeded at each dilution were checked and recorded every 12 hours. Finally, the death times of all chicken embryos seeded at the highest dilution ratio of the viruses are averaged as the MDT of the viruses in discussion.

### IV. Determination of ICPI

According to the OIE standard, the recombinant chimeric viruses or parental viruses were first subjected to serial 10-fold dilution with sterilized PBS, and then intracerebrally seeded to ten 1-day-old SPF chicks (50 μL for each). Observations were continuously conducted for 10 days to record the daily morbidity and mortality of the chicks, and the intracerebral pathogenicity index of the viruses was calculated according to the Reed and Muench method.

The results of the measured virulence and pathogenicity of the recombinant chimeric viruses in Example 3 were shown in Table 1. The ICPI value of the parental virus strain Anhinga was 1.47; the ICPI values of rlasota-HN, rlasota-P and rlasota-M were significantly lower than that of Anhinga, and were 0.05, 0.11, and 0.17 respectively; and the ICPI value of rlasota-F was 1.17, which is also lower than that of Anhinga. The results showed that, compared with the parental virus strain Anhinga, the HN, P, M and F replacements could reduce the pathogenicity of Anhinga in chickens. rlasota-F as a recombinant virus showed higher controllability and safety than Anhinga.

TABLE 1
Comparison of virus titer, virulence, and pathogenicity

| Virus | HA | TCID₅₀ | EID₅₀ | MDT | | ICPI |
|---|---|---|---|---|---|---|
| rlasota | 2⁹ | 2.09 × 10⁷ | 6.67 × 10⁹ | >120 | h | 0.00 |
| Anhinga | 2⁷ | 3.24 × 10⁷ | 4.27 × 10⁸ | 82 | h | 1.47 |
| rlasota-HN | 2⁷ | 1.13 × 10⁷ | 1 × 10⁹ | >120 | h | 0.05 |
| rlasota-P | 2⁹ | 1.1 × 10⁸ | 3.56 × 10⁹ | >120 | h | 0.11 |
| rlasota-F | 2⁹ | 1.62 × 10⁸ | 6.67 × 10⁹ | 96 | h | 1.17 |
| rlasota- M | 2⁹ | 1.16 × 10⁸ | 2.24 × 10⁹ | >120 | h | 0.17 |
| rlasota(F)-DR5 | 2⁹ | 3.67 × 10⁷ | 3.78 × 10⁹ | 94 | h | 1.11 |
| rlasota(F)-TRAIL | 2⁹ | 4.6 × 10⁷ | 2.29 × 10⁹ | 90 | h | 1.23 |
| rlasota(F)-hIL2 | 2¹⁰ | 1.71 × 10⁸ | 6.67 × 10⁹ | 96 | h | 1.07 |
| rlasota(F)-P53 | 2⁹ | 3.92 × 10⁷ | 3.37 × 10⁹ | 96 | h | 1.20 |
| rlasota(F)-PD1 | 2¹⁰ | 1.92 × 10⁸ | 4.92 × 10⁹ | 94 | h | 1.12 |
| rlasota(F)-CD | 2⁹ | 4.11 × 10⁷ | 2.12 × 10⁹ | 98 | h | 1.07 |
| rlasota(F)-mIL12 | 2⁹ | 3.16 × 10⁷ | 3.52 × 10⁹ | 98 | h | 1.06 |
| rlasota(F)-DR5-TRAIL | 2⁹ | 3.16 × 10⁷ | 3.61 × 10⁹ | 92 | h | 1.20 |
| rlasota(F)-mIL12-hIL2 | 2⁹ | 1.61 × 10⁸ | 4.67 × 10⁹ | 96 | h | 1.17 |
| rlasota(F)-P53-hIL2 | 2⁹ | 4.11 × 10⁷ | 3.92 × 10⁹ | 94 | h | 1.21 |
| rlasota(F)-PD1-hIL2 | 2¹⁰ | 1.58 × 10⁸ | 5 × 10⁹ | 98 | h | 1.11 |

### Example 4: Killing Effect of Recombinant Chimeric Virus Against Tumour Cells as Detected in Cell Fusion Experiment

The tumour cells refer to liver cancer cells HepG2, kidney cancer cells Caki-1, non-small cell lung cancer cells A549, melanoma cells SK-MEL-5, or neuroblastoma cells LN229.

I. Infection of Tumour Cells with Recombinant Chimeric Viruses

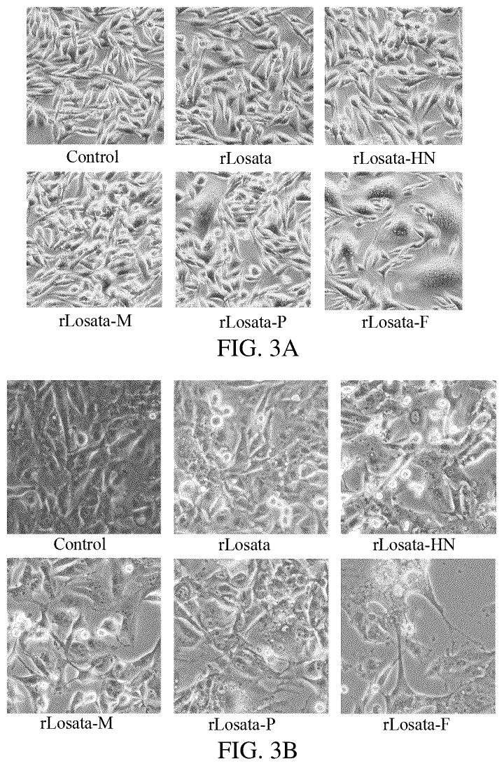

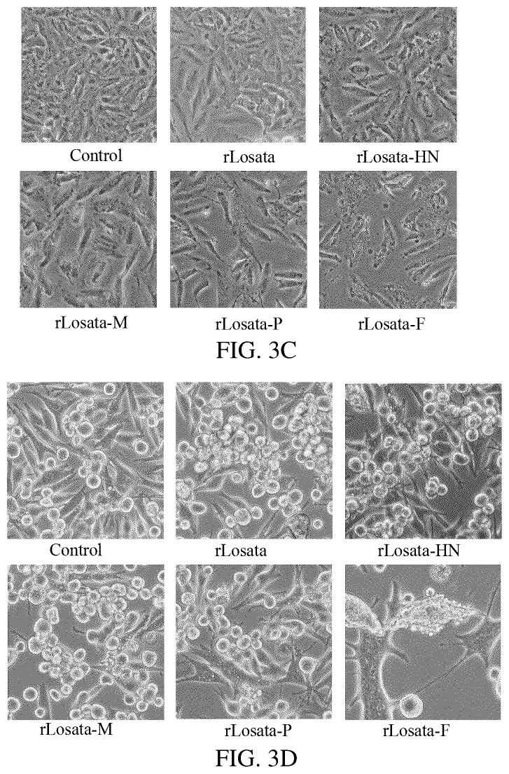

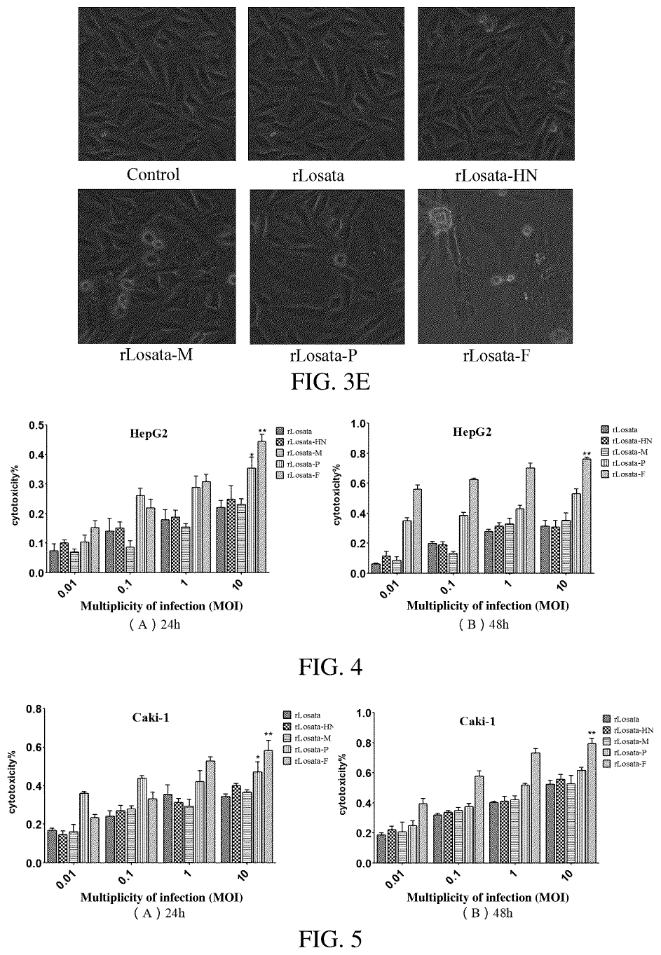

Tumour cells in the logarithmic growth phase were digested with trypsin and then collected, and seeded into a 6-well plate. The cells were infected with the recombinant chimeric viruses rlasota-HN, rlasota-F, rlasota-P, and rlasota-M at 1 MOI respectively, the parental virus rlasota was used as a control, and a control group was a virus-free SPF chicken embryo allantoic fluid. After 24 h, the 6-well plate was taken out, and observed under the inverted fluorescent microscope for a cell fusion effect. The results could be found in FIG. 3A to FIG. 3E.

The results showed that, compared with the parental viruses, the recombinant chimeric viruses rlasota-HN, rlasota-P, and rlasota-M produced more syncytia than rlasota, but without significant difference; and the chimeric virus rlasota-F produced more syncytia than rlasota, with an extremely significant difference.

### Example 5: Effect of Recombinant Chimeric Virus on Tumour Cells

The tumour cells refer to liver cancer cells HepG2, kidney cancer cells Caki-1, non-small cell lung cancer cells A549, melanoma cells SK-MEL-5, or neuroblastoma cells LN229.

I. Detection of inhibitory effect of recombinant chimeric virus on tumour cells by MTT method

Tumour cells in the logarithmic growth phase were digested by trypsin, and then, prepared into a cell suspension of 2×10⁴ cells/mL by using the DMEM or RPMI-1640 culture medium containing 10% calf serum. The cell suspension was seeded in a 96-well plate (200 μl per well), placed in a 5% CO2 environment at 37° ° C. for static culture for 24 h; the culture supernatant was discarded; and the residue was washed with a PBS buffer and then grouped. 100 μL of chimeric viruses at 0.01 MOI, 0.1 MOI, 1 MOI, and 10 MOI were added to each group of experimental wells, respectively; 100 μL of rlasota at 0.01 MOI, 0.1 MOI, 1 MOI, and 10 MOI was added to the control group, respectively; and 100 μL of DMEM was added to negative control wells. After 1 h of virus infection, the virus solution was discarded, 200 μL of PBS was added to each well for washing once; and a cell maintenance medium containing 5% serum was added for culture. After culturing for 24 h and 48 h respectively, 20 μL of MTT solution (5 mg/mL) was added to each well and incubated for 4 h; the culture solution was discarded; 150 μL of DMSO was added to each well; and after horizontal oscillating shake for 10 min, the OD value at 490 nm was measured with a microplate reader, and the cell growth inhibition rate was calculated.

Inhibition rate=(OD value of negative control treated well−OD value of experimental treated well)/OD value of negative control treated well×100%.

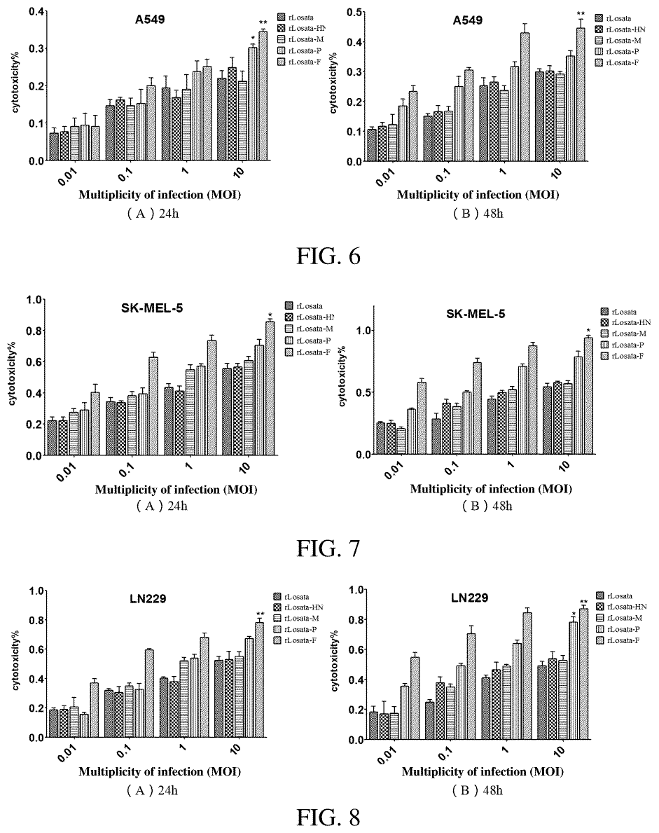

The inhibition rate against HepG-2 cells could be found in FIG. 4; the inhibition rate against Caki-1 cells could be found in FIG. 5; the inhibition rate against A549 cells could be found in FIG. 6; the inhibition rate against SK-MEL-5 cells could be found in FIG. 7; and the inhibition rate against LN229 cells could be found in FIG. 8.

The results showed that both the parental virus rlasota and the chimerical viruses rlasota-HN, rlasota-F, rlasota-P, and rlasota-M showed an inhibitory effect on the above cells; the inhibitory effect of the chimeric virus lasota-F is significantly higher than that of the parental virus rlasota; the inhibitory effects of other chimeric viruses rlasota-HN, rlasota-NP, and rlasota-M are higher than that of the parental virus rlasota, without significant difference; and the killing effects of the recombinant viruses on the tumour cells were positively correlated with time and dose.

### II. Killing Effect of Recombinant Chimeric Virus Against Liver Cancer Cells as Detected by Annexin V/PI Method

HepG2 tumour cells in the logarithmic growth phase were collected and prepared into a cell suspension of 1-10×10⁴ cells/mL (containing 0.001% trypsin). The cell suspension was mixed well and added to a 6-well plate, and after the cells grew to 60-80%, group experiments were carried out.

HepG2 cells added with the virus-free SPF chicken embryo allantoic fluid were taken as a null cell control group (control); HepG2 cells added with 1 MOI rlasota were taken as a control group; and HepG2 cells added with 1 MOI recombinant chimeric viruses were taken as experiment groups.

After 12 hours of infection, the cells (0.5-1×10⁶ cells) were digested with 0.25% trypsin, and washed twice with PBS; 500 μL of binding buffer was pipetted with a pipette to gently suspend the cells; 10 μL of FITC-labeled Annexin-V was added and mixed well; 5 μL of PI was then added and mixed well; immediately after 5-15 minutes of reaction in the dark, quantitative detection was carried out by flow cytometry; and at the same time, the HepG2 cells without AnnexinV-FITC and PI added were set up as a negative control.

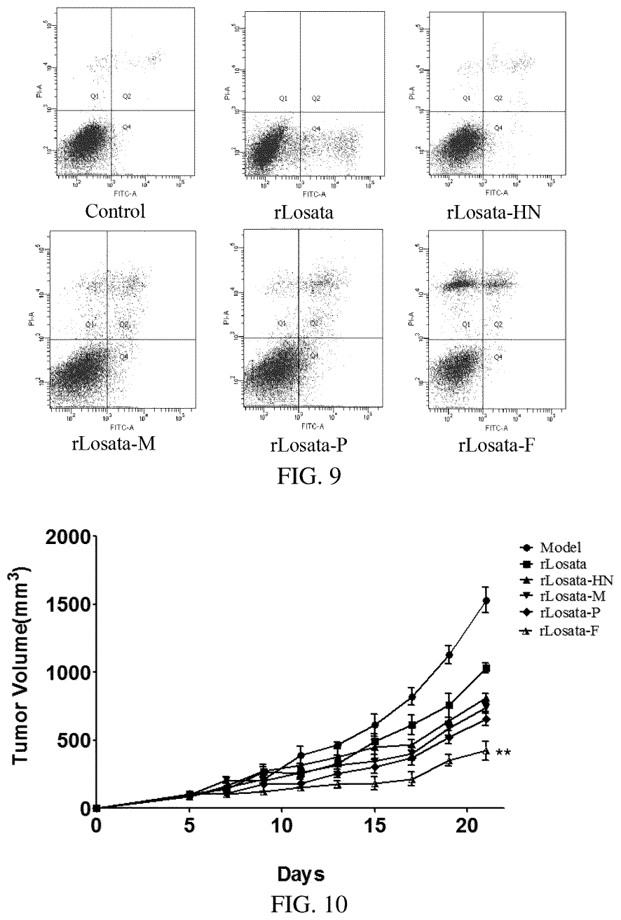

The results could be found in FIG. 9 and showed that both the parental viruses and the recombinant chimeric viruses could induce the apoptosis of tumour cells; the effect of the virus rlasota-F was significantly stronger than that of the virus rlasota; and the effects of other recombinant chimeric viruses were not significantly different from that of the virus rlasota.

### Example 6: Therapeutic Effect of Recombinant Chimeric Virus on Tumours and Virus Safety Detection

### I. Therapeutic Effect of Recombinant Chimeric Virus on Tumours

### 1. Establishment of Balb/c Mice H22 Liver Cancer Animal Model

Kunming mice of 6 weeks old were used for experiments. H22 mice liver cancer cells were seeded into the abdominal cavities of Humming mice, and after 7 days, the ascites occurred, and the mice were sacrificed by neck breakage. The ascites containing H22 cells was extracted under a sterile condition; a proper amount of PBS was added to formulate a cancer cell suspension; and the cells were counted and measured in viability, with the viable cell rate up to 95%. The cell density was adjusted to 106 cells/mL for later use. Subcutaneous injection was performed on each Balb/c mouse at the right groin at a dose of 0.2 mL, approximately containing 2×105 tumour cells. After 8-12 days, when a solid tumour with a diameter of 5-8 mm was formed, the modeling was successful, and the subsequent experiments could be carried out. The mice with the tumour diameter of 5-8 mm were selected, from which individuals with greatly varied forms and sizes were removed, and the remaining mice were taken as model mice.

### 2. Therapeutic Effect of Recombinant Chimeric Virus on Tumours

The model mice were randomly divided into six groups, with 15 mice in each group. They were treated as follows:
an rlasota-HN group: 0.2 mL of rlasota-HN virus solution (containing 10⁷ pfu viruses) prepared in Example 2 was injected into the tumours of the model mice every day for 14-day treatment;
an rlasota-F group: 0.2 mL of rlasota-F virus solution (containing 10⁷ pfu viruses) prepared in Example 2 was injected into the tumours of the model mice every day for 14-day treatment;
an rlasota-P group: 0.2 mL of rlasota-P virus solution (containing 10⁷ pfu viruses) prepared in Example 2 was injected into the tumours of the model mice every day for 14-day treatment;
an rlasota-M group: 0.2 mL of rlasota-M virus solution (containing 10⁷ pfu viruses) prepared in Example 2 was injected into the tumours of the model mice every day for 14-day treatment;
an rlasota group: 0.2 mL of rlasota virus solution (containing 10⁷ pfu viruses) prepared in Example 2 was injected into the tumours of the model mice every day for 14-day treatment;
an allantoic fluid group (Model): 0.2 mL of allantoic fluid was injected to the tumour of each model mice every day, and treated for 14 days.

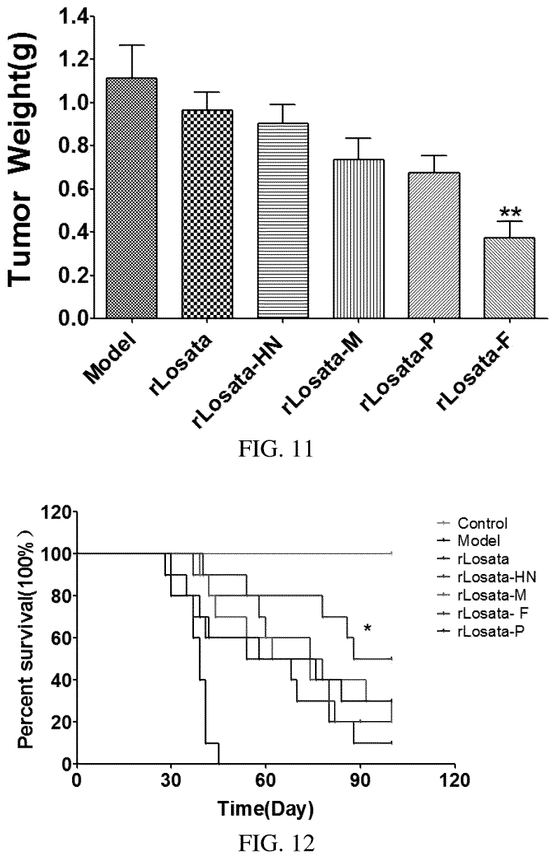

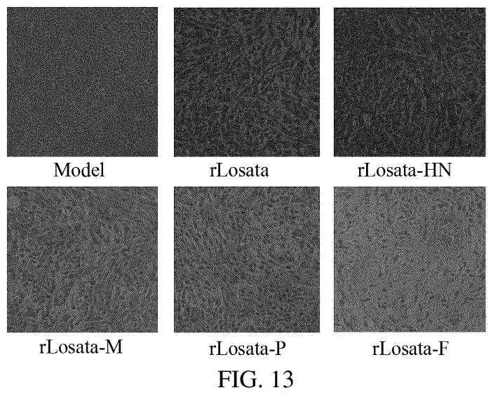

The tumour volume was measured every other day. After the treatment was completed, the mice were sacrificed, the tumours were removed and measured in terms of volume and weight (the results were shown in FIG. 10 and FIG. 11). The survival conditions of the remaining mice were observed after the treatment was completed, and the control group included normal mice without any treatment (FIG. 12). The results showed that, compared with the allantoic fluid group, both the parental viruses and the chimeric viruses showed significant inhibitory effects on the tumours; the anti-tumour effects of rlasota-P and rlasota-M were higher than that of the rlasota-HN group, without significant difference; and the inhibitory effect of rlasota-F is significantly higher than those of the above three types of chimeric viruses. Compared with the allantoic fluid group, both the parental viruses and the chimeric viruses could significantly improve the survival rate of the mice; and compared with the rlasota group, only rlasota-F could significantly improve the survival rate of the mice.

### 3. Observation of Pathological Sections of Tumours

To observe the tumour tissue cell morphology of NDV chimeric viruses and the control group after treatment, tumour tissues were acquired from each group of mice after treatment, and were prepared into tumour tissue paraffin sections following the conventional method. The morphological characteristics of tumour tissues in each treatment group were observed under a microscope, and then compared and analysed.

The results showed that after treatment, the tumour tissues from the model group were dense in structure with intact cell morphology and vigorous growth; and the tumour tissues from the rlasota virus control group undergone slight necrosis; the rlasota-HN, rlasota-P, and rlasota-M groups exhibited incomplete cell structure, accompanied with lymphocytes infiltration to a certain extent; and the rlasota-F group exhibited loose tumour tissues and incomplete cell structure, accompanied with massive lymphocytes infiltration. (See FIG. 13)

### II. Safety Detection of Recombinant Chimeric Viruses

### Acute Toxicity Test

Health Kunming mice of 4-6 weeks old were selected, 20 mice in total including half female and half male, and the mice were divided into two groups, 10 mice in each group. The mice in the control group were fed normally. Each mouse in the experiment groups was intraperitoneally injected with 1×10⁴ pfu of rlasota-F, and was observed for 48 hours after injection. If the mice under test experienced respiratory depression, unsteady limbs, paralysis symptoms, convulsions, fur trembles and other adverse reactions, and death, these mice were determined to be positive.

### Subacute Toxicity Test

Health Kunming mice of 4-6 weeks old were selected, 20 mice in total including half female and half male, and the mice were divided into two groups, 10 mice in each group. Each mouse in the experiment groups was intraperitoneally injected with 1×10⁴ pfu of rlasota-F, and the mice in the control group were fed normally. The responses of the mice were continuously recorded within 4 weeks, and the observations focused on aspects such as water intake, food intake, fur colour and body weight. If everything is normal without any adverse reaction or death, these mice were determined to be negative.

The results showed that the recombinant chimeric viruses had no adverse effect on the normal growth of mice, and were safe and reliable.

### Example 7: Effects of Recombinant Chimeric Viruses Expressing Exogenous Genes on Tumour Cells

The tumour cells included liver cancer cells HCCLM3, melanoma cells SK-MEL2, lung cancer cells A549, pancreatic cancer cells ASPC-1, thyroid cancer cells FTC133, kidney cancer cells 786-O, neuroblastoma cells U87-MG, myosarcoma cells HT1080, esophageal cancer cells KYSE-150, uterine cancer cells Hela, breast cancer cells MCF7, and colorectal cancer cells HCT116.

### I. Detection of Inhibitory Effect of Recombinant Chimeric Virus on Tumour Cells by MTT Method

Tumour cells in the logarithmic growth phase were digested by trypsin, and then prepared into a cell suspension at 2×10⁴ cells/mL by using a DMEM, F12 or RPMI-1640 culture medium containing 10% calf serum. The cell suspension was seeded in a 96-well plate (200 μl per well), placed in a 5% CO2 environment at 37° ° C. for static culture for 24 h; the culture supernatant was discarded; and the residue was washed with a PBS buffer and then grouped. 100 μL of 0.1 MOI chimeric viruses expressing exogenous genes were added to each group of experimental wells; 0.1 MOI rlasota-F was added to the control group; and 100 μL of DMEM was added to the negative control wells. After 1 h of virus infection, the virus solution was discarded, 200 μL of PBS was added to each well for washing once; and a cell maintenance medium containing 5% serum was added for culture. After culturing for 24 h, 20 μL of MTT solution (5 mg/mL) was added to each well and incubated for 4 h; the culture solution was discarded; 150 μL of DMSO was added to each well; and after horizontal oscillating shake for 10 min, the OD value at 490 nm was measured with a microplate reader, and the cell growth inhibition rate was calculated (the results were shown in Table 2).

$$\\ Inhibition rate = ( \begin{matrix}OD value of negative control treated well - \\ OD value of experimental treated well\end{matrix} ) / OD value of negative control treated well * 100 \% .$$

TABLE 2
Inhibitory effects of recombinant viruses expressing different
exogenous genes on different tumour cells (inhibition rate %)

| | | | | | | | | | rlasota | rlasota | rlasota | rlasota |
|---|---|---|---|---|---|---|---|---|---|---|---|---|
| | | rlasota | rlasota | rlasota | rlasota | rlasota | rlasota | rlasota | (F)- | (F)- | (F)- | (F)- |
| | rlasota- | (F)- | (F)- | (F)- | (F)- | (F)- | (F)- | (F)- | DR5- | mIL12- | P53- | PD1- |
| | F | DR5 | TRAIL | hIL2 | P53 | PD1 | CD | mIL12 | TRAIL | hIL2 | hIL2 | hIL2 |
| HCCLM3 | 27.39 | 32.55 | 27.50 | 42.13 | 35.83 | 32.43 | 30.17 | 33.43 | 55.56 | 57.73 | 50.95 | 55.04 |
| SK-MEL2 | 21.39 | 32.55 | 32.49 | 49.83 | 31.95 | 30.44 | 31.67 | 38.43 | 48.06 | 60.73 | 56.95 | 60.04 |
| U87-MG | 19.39 | 24.69 | 25.88 | 41.83 | 27.96 | 40.87 | 37.25 | 28.44 | 33.18 | 42.83 | 46.95 | 43.04 |
| 786-O | 15.89 | 19.56 | 17.83 | 26.84 | 22.95 | 23.43 | 31.25 | 23.46 | 38.18 | 42.84 | 36.97 | 38.56 |
| FITC133 | 18.89 | 21.55 | 20.34 | 26.84 | 35.45 | 28.43 | 21.25 | 22.93 | 33.78 | 38.23 | 34.45 | 33.04 |
| HT1080 | 13.47 | 18.33 | 19.62 | 25.98 | 23.46 | 25.87 | 32.25 | 32.93 | 29.12 | 31.88 | 39.95 | 35.64 |
| Hela | 11.96 | 28.09 | 17.84 | 30.22 | 10.45 | 12.86 | 9.63 | 15.67 | 33.10 | 32.83 | 24.95 | 29.94 |
| ASPC-1 | 11.22 | 19.84 | 17.11 | 21.99 | 29.66 | 15.49 | 16.62 | 19.79 | 26.81 | 25.77 | 32.54 | 24.87 |
| A549 | 15.41 | 17.26 | 18.98 | 16.14 | 26.22 | 22.39 | 20.16 | 25.9 | 24.69 | 28.12 | 29.95 | 30.66 |
| KYSE- | 11.28 | 15.04 | 17.56 | 24.65 | 17.45 | 14.31 | 15.09 | 21.93 | 25.18 | 30.56 | 26.03 | 21.87 |
| 150 | | | | | | | | | | | | |
| MCF7 | 9.8 | 17.63 | 16.56 | 25.66 | 14.45 | 29.74 | 15.09 | 14.43 | 21.89 | 26.31 | 25.53 | 33.28 |
| HCT116 | 12.25 | 15.58 | 19.52 | 13.16 | 16.08 | 26.57 | 20.02 | 18.93 | 22.59 | 27.09 | 29.12 | 33.96 |

The results showed that rlasota(F)-DR5, rlasota(F)-TRAIL, rlasota(F)-hIL2, rlasota(F)-P53, rlasota(F)-PD1, rlasota(F)-CD, rlasota(F)-mIL12, rlasota(F)-DR5-TRAIL, rlasota(F)-mIL12-hIL2, rlasota(F)-P53-hIL2 and rlasota(F)-PD1-hIL2a all showed inhibitory effects on the above cells, and showed higher inhibition rate against HCCLM3, SK-MEL2, FTC133, 786-O, U87-MG, and HT1080 than against A549, ASPC-1, KYSE-150, Hela, MCF7, and HCT116. Also, different virus strains did not exhibit completely identical cytotoxicity against A549, ASPC-1, KYSE-150, Hela, MCF7, and HCT116. Among these 12 types of tumour cells, rlasota(F)-DR5, rlasota(F)-TRAIL, and rlasota(F)-DR5-TRAIL showed higher inhibition rate against Hela cells; rlasota(F)-P53 and rlasota(F)-P53-hIL2 showed higher inhibition rate against A549 and ASPC-1 cells; rlasota(F)-hIL2, rlasota(F)-mIL12 and rlasota(F)-mIL12-hIL2 showed higher inhibition rate against A549, KYSE-150, and Hela cells; rlasota(F)-CD showed higher inhibition rate against A549 cells; and rlasota(F)-PD1 and rlasota(F)-PD1-hIL2 showed higher inhibition rate against A549, MCF7 and HCT116 cells.

### Example 8: Therapeutic Effect of Recombinant Chimeric Virus Expressing Exogenous Gene on Tumours

1. Balb/c 4T1 breast tumour-bearing mice were established according to the method described in Example 6.
2. Therapeutic effect of recombinant chimeric virus on tumours

The model mice were randomly divided into 13 groups, with 10 mice in each group. These groups were treated as follows respectively:

an rlasota(F)-DR5 group: 0.1 mL of rlasota(F)-DR5 virus solution (containing 5×10⁷ pfu viruses) prepared in Example 2 was injected into the tumours of the model mice every day for 21-day treatment;
an rlasota(F)-TRAIL group: 0.1 mL of rlasota(F)-TRAIL virus solution (containing 5×10⁷ pfu viruses) prepared in Example 2 was injected into the tumours of the model mice every day for 21-day treatment;
an rlasota(F)-hIL2 group: 0.1 mL of rlasota(F)-hIL2 virus solution (containing 5×10⁷ pfu viruses) prepared in Example 2 was injected into the tumours of the model mice every day for 21-day treatment;
an rlasota(F)-P53 group: 0.1 mL of rlasota(F)-P53 virus solution (containing 5×10⁷ pfu viruses) prepared in Example 2 was injected into the tumours of the model mice every day for 21-day treatment;
an rlasota(F)-PD1 group: 0.1 mL of rlasota(F)-PD1 virus solution (containing 5×10⁷ pfu viruses) prepared in Example 2 was injected into the tumours of the model mice every day for 21-day treatment;
an rlasota(F)-CD group: 0.1 mL of rlasota(F)-CD virus solution (containing 5×10⁷ pfu viruses) prepared in Example 2 was injected into the tumours of the model mice every day for 21-day treatment;
an rlasota(F)-mIL12 group: 0.1 mL of rlasota(F)-mIL12 virus solution (containing 5×10⁷ pfu viruses) prepared in Example 2 was injected into the tumours of the model mice every day for 21-day treatment;
an rlasota(F)-DR5-TRAIL group: 0.1 mL of rlasota(F)-DR5-TRAIL virus solution (containing 5×10⁷ pfu viruses) prepared in Example 2 was injected into the tumours of the model mice every day for 21-day treatment;
an rlasota(F)-mIL12-hIL2 group: 0.1 mL of rlasota(F)-mIL12-hIL2 virus solution (containing 5×10⁷ pfu viruses) prepared in Example 2 was injected into the tumours of the model mice every day for 21-day treatment;
an rlasota(F)-P53-hIL2 group: 0.1 mL of rlasota(F)-P53-hIL2 virus solution (containing 5×10⁷ pfu viruses) prepared in Example 2 was injected into the tumours of the model mice every day for 21-day treatment;
an rlasota(F)-PD1-hIL2 group: 0.1 mL of rlasota(F)-PD1-hIL2 virus solution (containing 5×10⁷ pfu viruses) prepared in Example 2 was injected into the tumours of the model mice every day for 21-day treatment;
an rlasota-F group: 0.1 mL of rlasota-F virus solution (containing 5×10⁷ pfu viruses) prepared in Example 2 was injected into the tumours of the model mice every day for 21-day treatment;
an allantoic fluid group (Model): 0.1 mL of allantoic fluid was injected to the tumour of each model mice every day, and treated for 21 days.

The tumour volume was measured every other day. After the treatment was completed, the mice were sacrificed, the tumours were removed and measured in weight (the results as shown in Table 3). The results showed that compared with the allantoic fluid group, rlasota-F and the recombinant chimeric viruses expressing the exogenous genes exhibited significant inhibitory effects on the tumours; the anti-tumour effects of rlasota(F)-DR5, rlasota(F)-TRAIL and rlasota(F)-CD are higher than that of the rlasota-F group, without significant difference; the anti-tumour effects of rlasota(F)-P53 and rlasota(F)-mIL12 are significantly higher than that of the rlasota-F treatment group; and the anti-tumour effects of rlasota(F)-hIL2 and rlasota(F)-PD1 showed extremely significant differences from the rlasota-F group. Compared with a single-gene treatment group, a dual-gene treatment group showed a further enhanced inhibitory effect against tumours. The rlasota(F)-mIL12-hIL2 and rlasota(F)-PD1-hIL2 groups exhibited the best effect, and the tumours in several mice almost regressed after the treatment was completed.

TABLE 3
Average volume and tumour weight of recombinant chimeric viruses
expressing exogenous genes after the completion of treatment

| | | Average | Average |
|---|---|---|---|
| | Average | volume | tumour |
| | volume | after the | weight after |
| | before | completion | the completion |
| | treatment | of treatment | of treatment |
| Model | 108.66 mm³ | 1886.53 mm³ | 1.33 g |
| rlasota-F | 123.30 mm³ | 1152.25 mm³ | 1.01 g |
| rlasota(F)-DR5 | 146.88 mm³ | 846.69 mm³ | 0.86 g |
| rlasota(F)-TRAIL | 116.88 mm³ | 759.84 mm³ | 0.79 g |
| rlasota(F)-hIL2 | 143.59 mm³ | 516.74 mm³ | 0.51 g |
| rlasota(F)-P53 | 142.58 mm³ | 607.42 mm³ | 0.62 g |
| rlasota(F)-PD1 | 133.55 mm³ | 508.665 mm³ | 0.46 g |
| rlasota(F)-CD | 107.38 mm³ | 992.64 mm³ | 0.94 g |
| rlasota(F)-mIL12 | 127.98 mm³ | 544.22 mm³ | 0.67 g |
| rlasota(F)-DR5-TRAIL | 115.26 mm³ | 462.54 mm³ | 0.44 g |
| rlasota(F)-mIL12-hIL2 | 113.73 mm³ | 309.82 mm³ | 0.25 g |
| rlasota(F)-P53-hIL2 | 127.29 mm³ | 401.58 mm³ | 0.38 g |
| rlasota(F)-PD1-hIL2 | 130.43 mm³ | 289.28 mm³ | 0.18 g |

### Example 9: Safety Detection of Recombinant Chimeric Viruses Expressing Exogenous Genes on Tumours

Healthy SPF Balb/c mice of 6 weeks old were selected and grouped, 10 mice in each group. The mice in the control group were fed normally. Each mouse in the experiment groups was intraperitoneally injected with 5×10⁸ pfu (10 times the therapeutic dose) recombinant chimeric virus (rlasota(F)-DR5, rlasota(F)-TRAIL, rlasota(F)-hIL2, rlasota(F)-P53, rlasota(F)-PD1, rlasota(F)-CD, rlasota(F)-mIL12, rlasota(F)-DR5-TRAIL, rlasota(F)-mIL12-hIL2, rlasota(F)-P53-hIL2, or rlasota(F)-PD1-hIL2), and then observed for 30 days. As a result, the mice with obvious adverse reactions such as listlessness occurred, ruffled fur and death were determined as positive. After the observation was completed, the serum of mice in each group was collected to detect AST, ALT and other related indicators.

The results showed that on the second day after injection, the fur of several mice in the experiment groups ruffled, and their food and water intake were not affected. After one week of continuous injections, the fur of the mice returned to normal; and after continuous observation for one month, none of the mice in each experimental group showed obvious adverse reactions such as listlessness, ruffled fur and death. After the observation was completed, the serum of mice in each group was collected to detect AST, ALT, BUN, CREA and other related indicators by ELISA. The results showed that compared with normal mice, the activities of aspartate aminotransferase (AST) and alanine aminotransferase (ALT) as well as the concentrations of blood urea nitrogen (BUN) and serum creatinine (CREA) in each experiment group slightly increased, without significant difference. The results indicated that the chimeric virus treatment did not induce liver and kidney damage and serious toxic or side effects.

Finally, it should be noted that the above-mentioned examples are obviously examples given for the purpose of clearly illustrating the present application, and are not intended to limit the embodiments. For a person skilled in the art, other different forms of changes or variations can also be made based on the above description. It is unnecessary or impossible to exhaustively list all the embodiments herein. Furthermore, any obvious changes or variations derived herefrom shall still fall within the protection scope of the present application.

1-12. (canceled)

13. A recombinant Newcastle disease virus, obtained by replacing an F protein of a Newcastle disease virus lasota with an F protein of a Newcastle disease virus mesogenic strain.

14. The recombinant Newcastle disease virus according to claim 13, wherein a coding DNA of an F protein of the recombinant Newcastle disease virus is as set forth in SEQ ID NO: 1 at positions 7274-8935 from a 5′-terminus.

15. The recombinant Newcastle disease virus according to claim 13, being obtained by replacing an F gene in a genome of the Newcastle disease virus lasota with an F gene of the Newcastle disease virus mesogenic strain, wherein
a DNA corresponding to the F gene of the recombinant Newcastle disease virus is as set forth in SEQ ID NO: 1 at the positions 7274-8935 from the 5′-terminus.

16. The recombinant Newcastle disease virus according to claim 13, wherein a DNA corresponding to a genome of the recombinant Newcastle disease virus further comprises an exogenous gene, which is selected from one or more of a group consisting of DR5, TRAIL, hIL2, P53, PD1, CD, and mIL12;
the DR5 is as set forth in SEQ ID NO: 2, the TRAIL is as set forth in SEQ ID NO: 3, the hIL2 is as set forth in SEQ ID NO: 4, the P53 is as set forth in SEQ ID NO: 5, the PD1 is as set forth in SEQ ID NO: 6, the CD is as set forth in SEQ ID NO: 7, and the mIL12 is as set forth in SEQ ID NO: 8; and
the exogenous gene is located between a P gene and an M gene of the recombinant Newcastle disease virus.

17. A recombinant plasmid, obtained by replacing an F gene in a pBrlasota plasmid, wherein the F gene of the recombinant plasmid is as set forth in SEQ ID NO: 1 at positions 7274-8935 from a 5′-terminus.

18. The recombinant plasmid according to claim 17, wherein the recombinant plasmid is a DNA molecule plasmid as set forth in SEQ ID NO: 1.

19. The recombinant plasmid according to claim 17, further comprising an exogenous gene, which is selected from one or more of a group consisting of DR5, TRAIL, hIL2, P53, PD1, CD, and mIL12, wherein
the DR5 is as set forth in SEQ ID NO: 2, the TRAIL is as set forth in SEQ ID NO: 3, the hIL2 is as set forth in SEQ ID NO: 4, the P53 is as set forth in SEQ ID NO: 5, the PD1 is as set forth in SEQ ID NO: 6, the CD is as set forth in SEQ ID NO: 7, and the mIL 12 is as set forth in SEQ ID NO: 8; and
the exogenous gene is located between a P gene and an M gene of a genome of the recombinant Newcastle disease virus.

20. A preparation method for a recombinant Newcastle disease virus, comprising transfecting the recombinant plasmid according to claim 5 to cells or cell line for culturing to obtain the recombinant Newcastle disease virus.

21. The preparation method according to claim 20, wherein
the recombinant plasmid according to claim 5 and a helper plasmid are co-transfected to cells or cell line for culturing; and
the cell is a mammalian cell.

22. Use of the recombinant Newcastle disease virus according to claim 13 or a recombinant plasmid obtained by replacing an F gene in a pBrLasota plasmid, wherein the F gene of the recombinant plasmid is as set forth in SEQ ID NO: 1 at positions 7274-8935 from a 5′-terminus in preparation of a drug having the following function(s) of (a) and/or (b) and/or (c):
(a) treating a tumour;
(b) inhibiting tumour cell proliferation; and
(c) killing tumour cells.

23. The use according to claim 22, wherein
the tumour is selected from one or more of a group consisting of a liver cancer, a breast cancer, a non-small cell lung cancer, a melanoma, a neuroblastoma, a lung cancer, a pancreatic cancer, a thyroid cancer, a kidney cancer, a glioma, a myosarcoma, an esophageal cancer, a uterine cancer, and a colorectal cancer; and
the tumour cell is selected from one or more of a group consisting of a liver cancer cell, a breast cancer cell, a non-small cell lung cancer cell, a melanoma cell, a neuroblastoma cell, a lung cancer cell, a pancreatic cancer cell, a thyroid cancer cell, a kidney cancer cell, a glioma cell, a myosarcoma cell, an esophageal cancer cell, a uterine cancer cell, and a colorectal cancer cell.

24. A drug, comprising
the recombinant Newcastle disease virus according to claim 13, and/or
a recombinant plasmid obtained by replacing an F gene in a pBrLasota plasmid, wherein the F gene of the recombinant plasmid is as set forth in SEQ ID NO: 1 at positions 7274-8935 from a 5′-terminus, wherein
the drug has the following function(s) of (a) and/or (b) and/or (c):
(a) treating a tumour;
(b) inhibiting tumour cell proliferation; and
(c) killing tumour cells.


SEQUENCE LISTING (8 sequences)

SEQ ID NO: 1 - DNA, 18391 bp, Artificial Sequence
  gene (7274)..(8935): NDV LaSota, NDV Anhinga
ctggcgtaatagcgaagaggcccgcaccgatcgcccttcccaacagttgcgtagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataagcatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaagcggccgctaatacgactcactatagggaccaaacagagaatccgtaagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactccgcacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcagacccaaggtccaactctccaagcggcaatcctctctcgcttcctcagccccactgaatgatcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccactgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagaaggatgtctacaagatcccagctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggccccaaaccccccaccggaaccccagcgcctctggtgctgatcgcccggaccgcgctggcgttgggctgtgtctgtccggcgggctctcttgacggcagacctcttgcagctgcagggattgtggtaacgagagataaagcagtcaatatatacacttcatctcaaacggggtcaatcatagtcaagttactcccaaatatgcccaaggacaaggaggcgtgcgcaaaagccccattagaggcrtacaatagaacactgaccactttactcactcctcttggygactccatccgsaggatacaagggtctgcaactacatctagaggaaggagacagaaacgttttgtaggtgctatcattggcagtatagctcttggggttgcgacagctgcacaagtaacagcagctgcagctctgatacaagccaaccagaacgctgccaacatcctccggcttaaggagagcattgctgcaaccaatgaagctgtgcacgaggtcactgacggattatcacaactagcgatggcgattgggaagatgcagcagtttgttaatgaccagtttaataatacggcgcgagaattggactgcatcaaaattacacaacaggttggtgtcgaactcaatttgtatctaactgaactgactacagtattcgggccacaaatcacttcccctgctttaactcagctaactatccaggcactttataatttagctggtggcaatatgaattacttattgactaagttaggtgtagggaacaatcaacttagctcattaattagtagtggcctgatcactggcaaccccattttatatgactcacagacccaactcttaggcatacagataaatgtaccctcagtcgggagcctaaataatatgcgtgccacctacttggagaccttatccgtaagcacaaccagggggttcgcctcagcacttgtcccgaaagttgtgacgcaagttggttctgtgatagaagaacttgacacctcatattgtatagaatctaatctggatttatattgtacaaggatagtgacattccccatgtctcccggcatttattcctgtctgagcggtaatacgtcagcttgtatgtattcaaagactgagggtgcactcactacaccatacatagctctcaagggctcagttattgctaattgcaagatgattacatgtagatgtgcagaccccccaggtatcatatcgcaaaattacggagaagctgtgtccctaatagataaacattcatgtaatgtcttatccctagacggaataaccctgaggctcagtggggaatttgatgcgacctatcaaaagaacatcttaatactagattcccaggtcatcgtgacaggcaatctcgatatatcaactgaacttgggaatgtcaacaactcgataagcagtgctctggacaaattagcggaaagtaacagcaagttaaacaaagtcaatgtcaacctaactagcacatctgctctcattacttatattgttctagctgtcatatctcttgttttcggcgtaattagcctgattctagcgtgctgcttgatgtataaacaaaaagcacaacaaaagaccttactatggcttgggaacaataccctcgatcagatgagagccaccacaaaaacatgaacacagatgaggaacgaaggtttccctaatagtaatttgtgtgaaagttctggtagtctgtcagttaagaaaaaatacgggtagaaggttaaccggcccgcggcgacgcgtggcctgagaggccttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggtaaataatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaataacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctactttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctagacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagtcccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagtgacctgacctcagataaagtagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcgaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggtttggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatcggccgatccggctgctaacaaagcccgaaaggaagctgagttggctgctgccaccgctgagcaataactagcataaccccttggggcctctaaacgggtcttgaggggttttttgctgaaaggaggaactatatccggatggccgccaccggtgggccttgcagcacatccccccttcgccag

SEQ ID NO: 2 - DNA, 1236 bp, Homo sapiens
  DR5
atggaacaacggggacagaacgccccggccgcttcgggggcccggaaaaggcacggcccaggacccagggaggcgcggggagccaggcctgggctccgggtccccaagacccttgtgctcgttgtcgccgcggtcctgctgttggtctcagctgagtctgctctgatcacccaacaagacctagctccccagcagagagcggccccacaacaaaagaggtccagcccctcagagggattgtgtccacctggacaccatatctcagaagacggtagagattgcatctcctgcaaatatggacaggactatagcactcactggaatgacctccttttctgcttgcgctgcaccaggtgtgattcaggtgaagtggagctaagtccctgcaccacgaccagaaacacagtgtgtcagtgcgaagaaggcaccttccgggaagaagattctcctgagatgtgccggaagtgccgcacagggtgtcccagagggatggtcaaggtcggtgattgtacaccctggagtgacatcgaatgtgtccacaaagaatcaggcatcatcataggagtcacagttgcagccgtagtcttgattgtggctgtgtttgtttgcaagtctttactgtggaagaaagtccttccttacctgaaaggcatctgctcaggtggtggtggggaccctgagcgtgtggacagaagctcacaacgacctggggctgaggacaatgtcctcaatgagatcgtgagtatcttgcagcccacccaggtccctgagcaggaaatggaagtccaggagccagcagagccaacaggtgtcaacatgttgtcccccggggagtcagagcatctgctggaaccggcagaagctgaaaggtctcagaggaggaggctgctggttccagcaaatgaaggtgatcccactgagactctgagacagtgcttcgatgactttgcagacttggtgccctttgactcctgggagccgctcatgaggaagttgggcctcatggacaatgagataaaggtggctaaagctgaggcagcgggccacagggacaccttgtacacgatgctgataaagtgggtcaacaaaaccgggcgagatgcctctgtccacaccctgctggatgccttggagacgctgggagagagacttgccaagcagaagattgaggaccacttgttgagctctggaaagttcatgtatctagaaggtaatgcagactctgccatgtcctaa

SEQ ID NO: 3 - DNA, 624 bp, Homo sapiens
  TRAIL
atggagacagacacactcctgctatgggtactgctgctctgggttccaggatccactggtacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaa

SEQ ID NO: 4 - DNA, 462 bp, Homo sapiens
  IL2
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttaa

SEQ ID NO: 5 - DNA, 1182 bp, Homo sapiens
  P53
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactga

SEQ ID NO: 6 - DNA, 504 bp, Homo sapiens
  PD1
atggagacagacacactcctgctatgggtactgctgctctgggttccaggatccactggtccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaatga

SEQ ID NO: 7 - DNA, 1284 bp, Escherichia coli
  CD
atgtcgaataacgctttacaaacaattattaacgcccggttaccaggcgaagaggggctgtggcagattcatctgcaggacggaaaaatcagcgccattgatgcgcaatccggcgtgatgcccataactgaaaacagcctggatgccgaacaaggtttagttataccgccgtttgtggagccacatattcacctggacaccacgcaaaccgccggacaaccgaactggaatcagtccggcacgctgtttgaaggcattgaacgctgggccgagcgcaaagcgttattaacccatgacgatgtgaaacaacgcgcatggcaaacgctgaaatggcagattgccaacggcattcagcatgtgcgtacccatgtcgatgtttcggatgcaacgctaactgcgctgaaagcaatgctggaagtgaagcaggaagtcgcgccgtggattgatctgcaaatcgtcgccttccctcaggaagggattttgtcgtatcccaacggtgaagcgttgctggaagaggcgttacgcttaggggcagatgtagtgggggcgattccgcattttgaatttacccgtgaatacggcgtggagtcgctgcataaaaccttcgccctggcgcaaaaatacgaccgtctcatcgacgttcactgtgatgagatcgatgacgagcagtcgcgctttgtcgaaaccgttgctgccctggcgcaccatgaaggcatgggcgcgcgagtcaccgccagccacaccacggcaatgcactcctataacggggcgtatacctcacgcctgttccgcttgctgaaaatgtccggtattaactttgtcgccaacccgctggtcaatattcatctgcaaggacgtttcgatacgtatccaaaacgtcgcggcatcacgcgcgttaaagagatgctggagtccggcattaacgtctgctttggtcacgatgatgtcttcgatccgtggtatccgctgggaacggcgaatatgctgcaagtgctgcatatggggctgcatgtttgccagttgatgggctacgggcagattaacgatggcctgaatttaatcacccaccacagcgcaaggacgttgaatttgcaggattacggcattgccgccggaaacagcgccaacctgattatcctgccggctgaaaatgggtttgatgcgctgcgccgtcaggttccggtacgttattcggtacgtggcggcaaggtgattgccagcacacaaccggcacaaaccaccgtatatctggagcagccagaagccatcgattacaaacgttga

SEQ ID NO: 8 - DNA, 1623 bp, Mus musculus
  IL12
atgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaagctgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccgttcctggagtaggggtacctggagtgggcggatctatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagtccccccgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcctag